(19) United States
(12) Patent Application Publication
(10) Pub. No.: US 2026/0191598 A1
(43) Pub. Date: Jul. 9, 2026

(54) SURGICAL GUIDANCE AND NAVIGATION SYSTEM

(71) Applicant: Surgical Targeted Solutions Inc., Akron, OH (US)

(72) Inventors: Ian Kay, Medina, OH (US); Joshua P. Morgan, Lebanon, OH (US)

(21) Appl. No.: 19/439,114

(22) Filed: Jan. 2, 2026

Related U.S. Application Data
(60) Provisional application No. 63/741,657, filed on Jan. 3, 2025.

(51) Int. Cl. A61B 34/20; A61B 34/10; A61B 34/30; A61B 90/00; A61B 90/10

(52) U.S. Cl. CPC A61B 34/20; A61B 34/10; A61B 34/30; A61B 90/10; A61B 90/39; A61B 2034/107; A61B 2034/2059; A61B 2034/2072; A61B 2034/301; A61B 2090/363; A61B 90/37; A61B 2090/376; A61B 2090/3966

(57) ABSTRACT

A stereotactic surgical guidance and navigation system for surgical procedures, such as orthopedic surgical procedures. The system can be used as an intraoperative planning and navigation system compatible with commonly used surgical tooling (e.g., guide wires, or “k-wires”) in open and percutaneous orthopedic surgical procedures. The system registers the patient's anatomy to a coordinate system, continuously tracks the patient's anatomy and the tooling positions, and visually presents the real-time entry point, trajectory, and insertion depth of the tooling in relation to the anatomy. By monitoring the visual displays, the surgeon is able to refine the projected final tooling placement before inserting the navigated tooling into the patient's anatomy.

### CROSS-REFERENCE TO RELATED APPLICATIONS

This application claims the benefit of U.S. Provisional Application No. 63/741,657, filed Jan. 3, 2025, the entire disclosure of which is incorporated herein by reference for all purposes.

### TECHNICAL FIELD

This application relates to a medical device for surgical assistance, and more particularly, to a surgical guidance and navigation system. Even more specifically, the application relates to a stereotactic surgical guidance and navigation system for use with surgical tooling and instruments during surgery, such as in open and percutaneous orthopedic surgical procedures.

### BACKGROUND

Many orthopedic procedures require cutting or drilling into bone. For example, bone drilling is a fundamental technique for fracture repair and implant placement, both of which are medical processes to provide structural support to a weakened, diseased, damaged or broken bone segment. The bone drilling creates holes for inserting screws or other implants to fix bone fractures, stabilize joints, or place devices like prosthetic components. In some cases, the bone drilling creates a pathway for other treatments such as for the delivery of bone hardening materials, or chemotherapy and pharmaceutical agents. Like many invasive medical techniques for neurosurgery and otolaryngology, bone drilling is an orthopedic surgical procedure that is extremely delicate and requires precision and accuracy in order to drill only to the desired depth and location within the bone, since even the smallest deviation can have significant consequences. For at least this reason, surgical guidance and navigation assistance is desirable.

Surgical navigation systems represent a significant advancement in modern medicine as it provides surgeons with the tooling needed for enhanced precision and improved outcomes in complex surgical procedures like the one described above. Today, numerous types of surgical instrument guidance and navigation systems exist that attempt to address this need. Many of these systems operate in a manner similar to GPS systems, providing guidance to the surgeon through intricate anatomy of the human body to accurately target specific areas while avoiding critical structures. Central to the surgical navigation system is the combination of advanced imaging technologies (e.g., CT, MRI, or fluoroscopy) and real-time tracking mechanisms in order to provide a detailed map of the patient's anatomy to be used to plan and execute the surgery with accuracy. The surgeon uses this map to navigate instruments, such as a K-wire and bone drill, through the body. Often, a computer screen displays the precise location of the instruments in relation to the patient's anatomy.

Perhaps the most accurate of these types of surgical navigation systems are the robotic guidance systems, which utilize advanced technologies to allow for pre-operative or intraoperative planning. In some robotic systems, they also control and direct the movement of robotic arms or fingers that hold the surgical instruments. These robotic guidance systems provide intelligent services and information by interacting with their environment through the use of various sensors, actuators and human interfaces. In many cases using robotic guidance systems can provide benefits such as increased precision, consistency and reproducibility, reduced operating time, and potential for faster patient recovery.

Although providing significant surgical benefits, many robotic guidance systems, particularly fully robotic systems having capacity to perform some or all of the surgery, come with high monetary and physical costs. These robotic guidance systems are very expensive and take up a large footprint in the operating room, due to the large housing they need to store the complex machinery that runs the systems. This creates large drawbacks where money or space may be limited, or in situations where there is high volume demand. Additionally, many of these large, fully robotic systems have very little mobility and portability, making it challenging to adapt to changing environments.

Less expensive (which can often mean correspondingly less complex and advanced) and less bulky surgical navigation systems have also been developed, including some which are hand-held instruments with incorporated navigation features such as movement sensors that interact with a computer screen so that the surgeon can track movement of the instrument intraoperatively. However, these systems lack the accuracy and precision of their larger, robotic counterparts. While these less complex systems still provide an effective option to assist the surgeon relying solely on hand-eye coordination, the need for an improved surgical guidance and navigation system still exists.

Accordingly, it would be desirable to provide a very precise and accurate surgical guidance and navigation system that is more affordable than a fully robotic system, and which has a smaller footprint to avoid creating an obstacle to the surgical viewing field or within the operating room for the surgeon. It is additionally desirable for such as system to have greater mobility allowing for increased positioning capabilities and adaptability to changing work environments. It would further be desirable to provide such a system that is easy to learn and implement with existing surgical tooling and instruments commonly found in an operating room.

### SUMMARY

The following presents a simplified summary of the claimed subject matter in order to provide a basic understanding of some aspects of the claimed subject matter. This summary is not an extensive overview of the claimed subject matter. It is intended to neither identify key or critical elements of the claimed subject matter nor delineate the scope of the claimed subject matter. Its sole purpose is to present some concepts of the claimed subject matter in a simplified form as a prelude to the more detailed description that is presented later.

The present system addresses the aforementioned unmet needs by providing a stereotactic surgical guidance and navigation system for surgical procedures, such as orthopedic surgical procedures. The system can be used as an intraoperative planning and navigation system compatible with commonly used surgical tooling (e.g., guide wires, or “k-wires”) in open and percutaneous orthopedic surgical procedures. The system registers the patient's anatomy to a coordinate system, continuously tracks the patient's anatomy and the tooling positions, and visually presents the real-time entry point, trajectory, and insertion depth of the tooling in relation to the anatomy. By monitoring the visual displays, the surgeon is able to refine the projected final tooling placement before and while inserting the navigated tooling into the patient's anatomy.

The present system provides a more precise and accurate surgical guidance and navigation system that is more affordable than a fully robotic system, has a smaller footprint to avoid creating an obstacle to the surgical viewing field or within the operating room for the surgeon, and has greater mobility allowing for increased adaptability and positioning. The system is also easy to learn and implement with existing surgical tooling and instruments commonly found in an operating room, such as a guidewire, k-wire, or drill.

In one embodiment, a surgical guidance and navigation system for tracking a tooling position relative to a patient is provided. The system can comprise a frame module housing a computerized subsystem including a computer module therein, and having electronically connected thereto a visual display; an articulating positioning arm connected to the frame module; a localized base module in communication with the frame module; and first and second kinematic tracking arms attached to the localized base module, the first kinematic tracking arm being configured for connection to a patient's bone for patient tracking, and the second kinematic tracking arm being configured to receive a surgical tooling for tooling tracking.

In some embodiments, the frame module may serve as a rolling cart or console having locking wheels along its base in contact with a floor of the operating room. In such embodiments, the frame module and any attached components may become mobile by unlocking the wheels and applying force to the unit. The wheels may additionally be locked to provide an immobile, stable work environment. In some embodiments, the articulating positioning arm extends from a center of the frame module. In other embodiments, the articulating positioning arm extends from a side of the frame module. In embodiments, the frame module is positioned on a non-operative side of the patient to avoid obstruction of a surgeon.

The localized base module can be attached to the articulating positioning arm in one embodiment. In such an embodiment the localized base module is physically mounted on the articulating positioning arm and is configured to connect to the computerized subsystem of the frame module. In another embodiment, the localized base module may be connected to another machine or equipment, including for example, the OR table. In certain embodiments the localized base module may be configured to serve as a reference coordinate frame to which all other tracked entities are mapped. In some embodiments the localized base module is positioned anterior to the patient to avoid the surgical field and other operating room equipment. In some embodiments, this can be done by flexibly positioning the articulating positioning arm.

The localized base module can include a mounting and communication interface configured to receive the first and second kinematic tracking arms. In one embodiment, the first and second kinematic tracking arms can be removably attachable to the mounting and communication interface of the localized base module. In another embodiment, the kinematic tracking arms can be fixedly attached to the mounting and communication interface of the localized base module. In embodiments, each kinematic tracking arm is attached to a mating socket on the localized base module and has freedom to move about the socket.

The first and second kinematic tracking arms can be configured for sensor-based continuous tracking. For example, the first and second kinematic tracking arms can each include an electromechanical linkage that allows for 6 degrees of freedom movement of an attached end effector. In embodiments the end effectors may be attached at a distal end of the kinematic tracking arms. The kinematic tracking arms can additionally track the position of the patient and tooling. In embodiments, the kinematic tracking arms may include encoders to allow for accurate and precise measurement of joint angles of each link of the kinematic tracking arms. The data generated from these encoders may be combined with known geometries of arm segments of the kinematic tracking arms as well as any attached components to allow for a continuous determination of a position in space of an end effector of the kinematic tracking arms. This determination may be made by using forward-kinematic calculations.

The system can further include a radiographic fiducial module for connection with one of the first or second kinematic tracking arms. In one embodiment, the radiographic fiducial module can be configured to attach to the patient's bone, and thus provide a dual function of also acting as a patient interface module. In another embodiment, the system further includes a patient interface module that is a separate component than the radiographic fiducial module. The patient interface module can connect to one of the first or second kinematic tracking arms and may further be configured to attach to the patient's bone. In embodiments, the radiographic fiducial module includes an array of radiopaque markers arranged in a geometric configuration. These radiopaque markers may be detectable by an image analysis algorithm implemented in a software application.

The system can further include a tooling guide module for connection to the second kinematic tracking arm. The tooling guide module can be configured to receive a surgical tooling, such as for example, a guidewire, drill bit, reamer, or saw blade. In some embodiments, the tooling guide module can include a depth-tracking sensor for determining a translation depth of the guidewire. The tooling guide module can include, for example, a guide pin depth tracking encoder, a microprocessor, or a zero depth indicator. The tooling guide module can also include a handle portion to allow manipulation of the position and trajectory of a tooling received within the tooling guide module. In embodiments, the tooling guide module establishes a geometric relationship between the kinematic tracking arm and the surgical tooling. In such embodiments, the precise axis of a surgical tool, for example a guide wire, inside the tooling guide module can be known in 3-dimensional space due to the mechanical connections between components.

In the present system, the first and second kinematic tracking arms are independently movable with respect to one another.

In some embodiments, the system may also include a distortion correction module. In certain embodiments, the distortion correction module may contain an array of radiopaque points arranged in a geometric configuration that may be visible in captured images. In various embodiments, the distortion correction module may be configured to connect to a C-arm fluouroscopic imager.

The system may be configured to provide real-time insertion profile projection of the tooling into a patient's bone on the visual display.

The present system may be used in a method of performing orthopedic surgery, and specifically for inserting a guidewire into a patient's bone. The system may be used to determine a desired depth and trajectory of the guidewire into the patient's bone while viewing the real-time insertion profile projection of the guidewire on the visual display.

In accordance with one aspect of the present disclosure, a method of performing orthopedic surgery using the surgical guidance and navigation system is provided. The method may include, attaching the localized base module to the articulating positioning arm; attaching the first and second kinematic tracking arms to a mounting and communication interface of the localized base module; attaching a tooling guide module to a distal end of the second kinematic tracking arm; inserting a surgical tool into the tooling guide module; attaching a radiographic fiducial model and a patient interface module to a distal end of the first kinematic tracking arm; positioning the frame module relevant to a patient prepared for surgery; positioning the articulating positioning arm such that the localized base module is located anterior to the patient; activating the computerized subsystem of the frame module; initiating a startup calibration routine to establish a baseline and repeatable initial position; securing the patient interface module to a bone of the patient; capturing fluouroscopic images; tracking the kinematic tracking arms and at least one end effector using known geometries and precise measurements of at least one joint angle of each arm to conduct forward kinematics calculations; detecting a radiopaque marker of the radiographic fiducial model to provide an image of a surgical worksite; measuring a translation depth of the surgical tool with a depth tracking sensor installed in the tooling guide module; and providing a real-time presentation of an insertion profile of the surgical tool.

In some embodiments, a step of conducting pre-operative CT imaging may be undertaken prior to attaching the localized base module to the articulating positioning arm.

In some embodiments, the step of measuring a translation depth of the surgical tool may further comprise viewing an image depicting a position of the surgical tool in 3-dimensional space displayed on the visual display of the frame module.

In some embodiments, the method may further comprise the step of utilizing a distortion correction module to correct fluoroscopic image distortion.

It is to be understood that both the foregoing general description and the following detailed description are exemplary and explanatory only and are not restrictive of the disclosure. Additional features of the disclosure will be set forth in part in the description which follows or may be learned by practice of the disclosure.

### BRIEF DESCRIPTION OF THE DRAWINGS

The accompanying drawings, which are incorporated in and constitute a part of this specification, illustrate several embodiments of the disclosure and together with the description, serve to explain the principles of the disclosure.

FIG. 1 illustrates a perspective view of an exemplary embodiment of a surgical guidance and navigation system in use.

FIG. 2A illustrates another perspective view of the system of FIG. 1.

FIG. 2B illustrates the system of FIG. 2A with attached kinematic tracking arms.

FIG. 3A illustrates a perspective view of an exemplary embodiment of a localized base module with attached kinematic tracking arms for use with the system of FIG. 1.

FIG. 3B illustrates the localized base module of FIG. 3A attached to the system of FIG. 1.

FIGS. 3C and 3D illustrate the system of FIG. 3B with attached kinematic tracking arms.

FIGS. 4A, 4B, 4C and 4D illustrate exemplary embodiments of a patient interface and radiographic fiducial module of the system of FIG. 1 in use.

FIGS. 5A and 5B illustrate perspective views of an exemplary embodiment of a tooling guide module of the system of FIG. 1 in use.

FIG. 6 illustrates a perspective view of an exemplary embodiment of a distortion correction module useful with the system of FIG. 1 in use.

FIG. 7 represents an exemplary embodiment of a surgical workflow process for using the system of FIG. 1.

### DESCRIPTION OF THE EMBODIMENTS

This description and the accompanying drawings illustrate exemplary embodiments and should not be taken as limiting, with the claims defining the scope of the present disclosure, including equivalents. Various mechanical, compositional, structural, and operational changes may be made without departing from the scope of this description and the claims, including equivalents. In some instances, well-known structures and techniques have not been shown or described in detail so as not to obscure the disclosure. Like numbers in two or more figures represent the same or similar elements. Furthermore, elements and their associated aspects that are described in detail with reference to one embodiment may, whenever practical, be included in other embodiments in which they are not specifically shown or described. For example, if an element is described in detail with reference to one embodiment and is not described with reference to a second embodiment, the element may nevertheless be claimed as included in the second embodiment. Moreover, the depictions herein are for illustrative purposes only and do not necessarily reflect the actual shape, size, or dimensions of the system or illustrated components.

It is noted that, as used in this specification and the appended claims, the singular forms “a,” “an,” and “the,” and any singular use of any word, include plural referents unless expressly and unequivocally limited to one referent. As used herein, the term “include” and its grammatical variants are intended to be non-limiting, such that recitation of items in a list is not to the exclusion of other like items that can be substituted or added to the listed items.

For purposes of illustration, the embodiments are described with reference to orthopedic tooling, instruments or implants in a surgical orthopedic procedure. However, the embodiments may be implemented in any surgical procedure requiring precise guidance and navigation including, such as for example, dental surgery, neurosurgery or surgery for otolaryngology.

As mentioned above, there is a need for improvements in surgical guidance and navigation systems. One such improved surgical guidance and navigation system providing improvements in affordability, size, mobility, and accessibility is provided herein below.

### Overview of the System

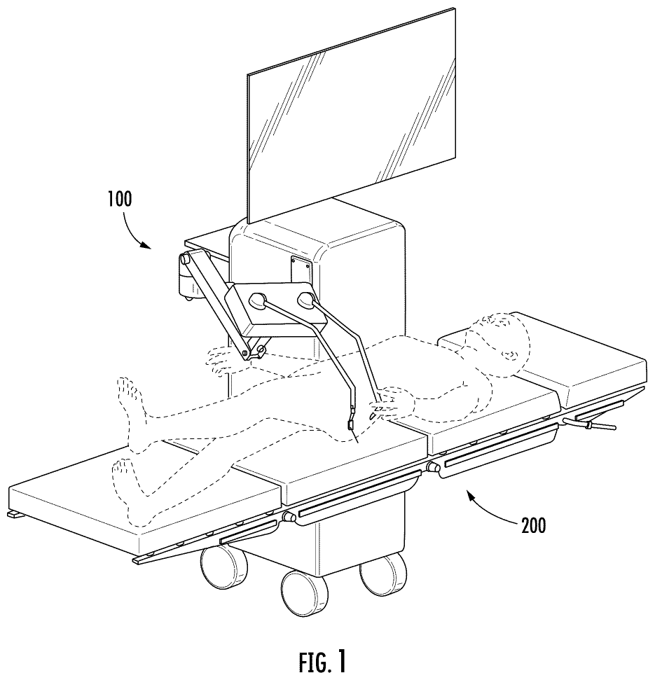

As shown in FIG. 1, an exemplary embodiment of a stereotactic surgical guidance and navigation system 100 (the “System”) for surgical procedures, such as for example orthopedic surgical procedures, is placed next to a representative orthopedic fracture table 200. The System 100 can be used as an intraoperative planning and navigation system compatible with commonly used surgical tooling (e.g., guide wires, or “k-wires”) in open and percutaneous orthopedic surgical procedures. In one embodiment, the System 100 registers the patient's anatomy to a coordinate system, continuously tracks the patient's anatomy and the tooling positions, and visually presents the real-time entry point, trajectory, and insertion depth of the tooling in relation to the anatomy. By monitoring the visual displays, the surgeon is able to align the projected tooling trajectory before inserting the navigated tooling into the patient's anatomy.

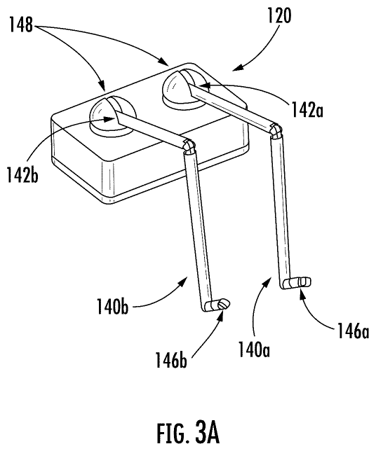

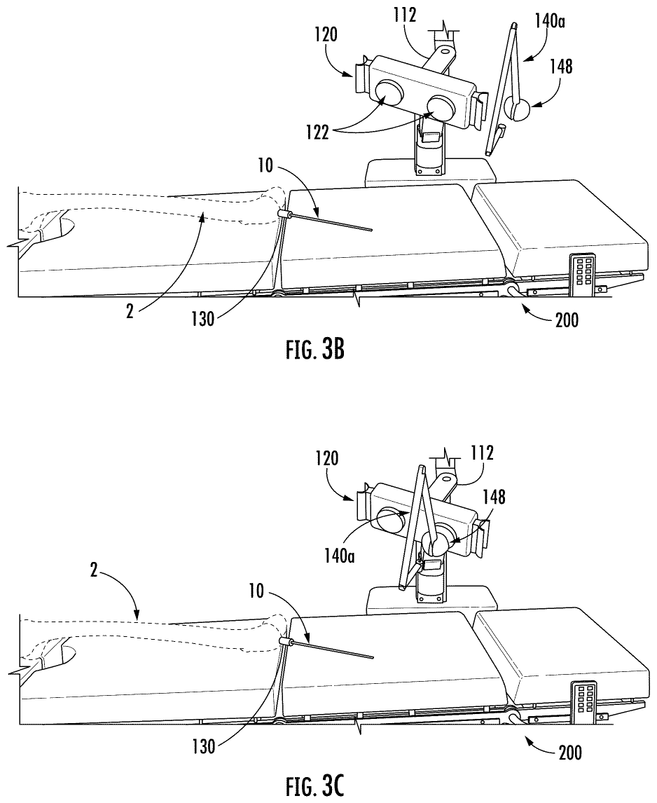

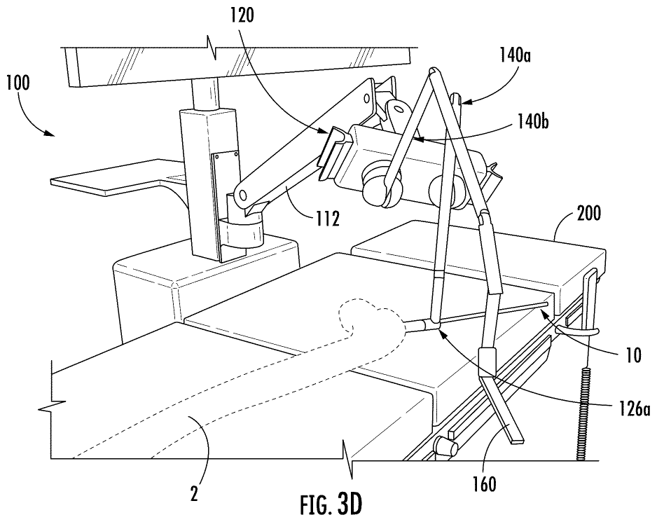

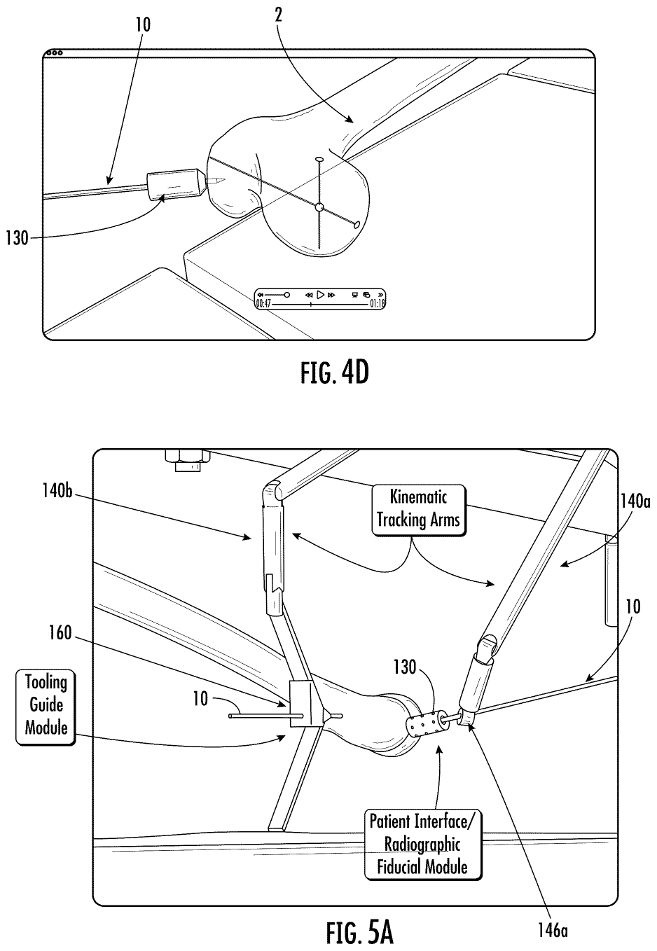

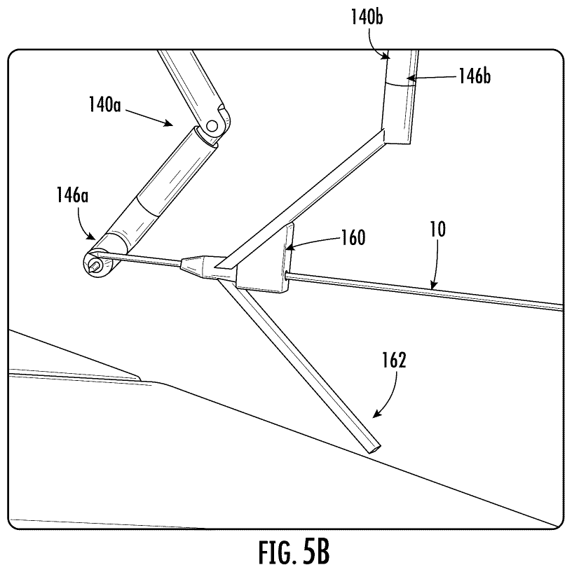

As mentioned above, the System 100 provides the advantage of being a precise and accurate surgical guidance and navigation system that is more affordable than a fully robotic system, and which has a smaller footprint to avoid creating an obstacle to the surgical viewing field or within the operating room for the surgeon. The System 100 also provides the advantage of being mobile as well as easy to learn and implement with existing surgical tooling commonly found in an operating room, such as for example a k-wire 10 as depicted in FIGS. 3B-3D, 5A and 5B.

Specific components, features and methods of using the System 100 will be described in greater detail below.

### Tracking Methodology

To enable intraoperative guidance to the surgeon while using the surgical tooling, it is necessary for the System 100 to continuously determine the positional relationship between the patient's anatomy and the surgical tooling during use. At a high level, this is accomplished using a combination of:
1) components with known (static) geometries (controlled through the system's design & manufacturing);
2) continuously measured pose changes (position & orientation) using Kinematic Tracking Arms (KTAs) designed to provide the required tracking resolution and accuracy;
3) calibration methods, during either the system's manufacturing processes or as a part of the required system setup workflow, to establish a positional baseline from which pose changes are continuously measured; and
4) fluoroscopic imaging in which radiopaque system components of known geometries are captured. These components facilitate distortion correction and provide geometric references, allowing the registration of the patient's anatomy into the system coordinate frame.

### Frame Module and Articulating Positioning Arm

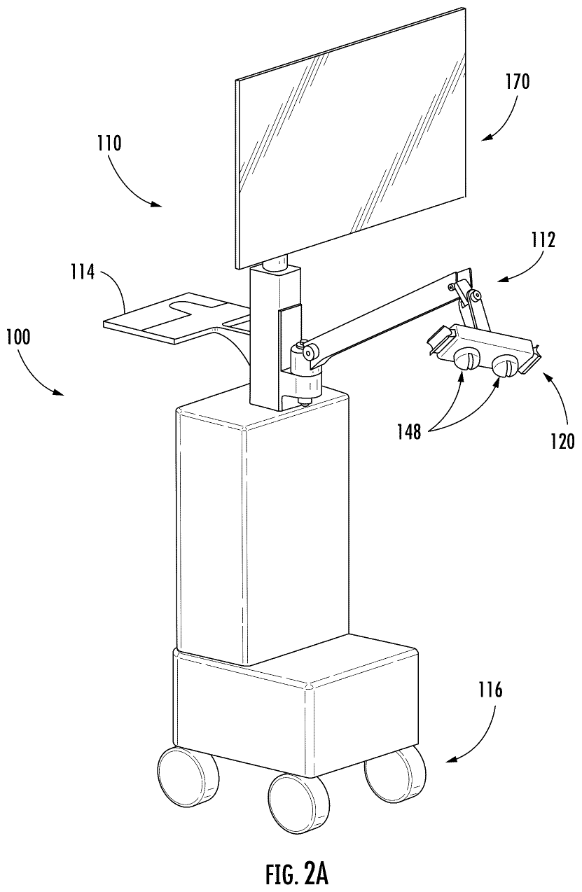

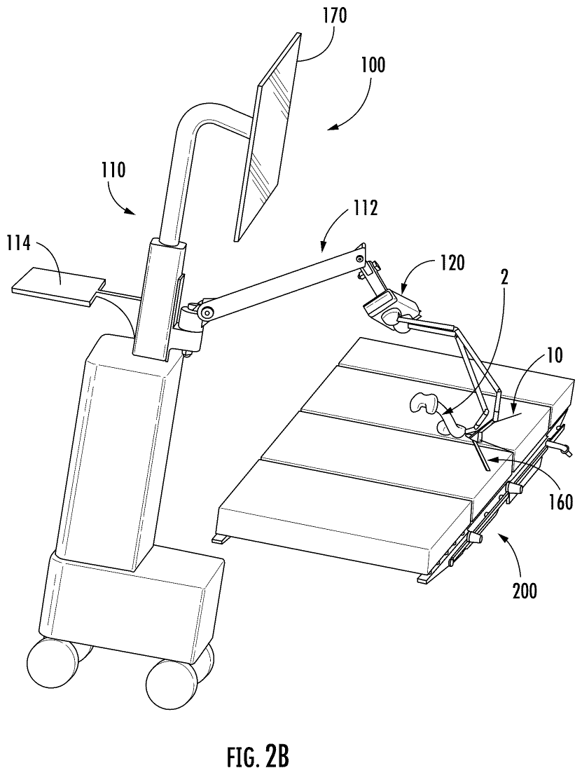

As shown in FIGS. 2A and 2B, the system comprises a Frame Module 110. The Frame Module 110 serves as a base unit that can house a computerized subsystem and/or provide a ledge or platform 114 for placement of a laptop, tablet, mobile phone or other electronic device for running a computerized operating system, as well as provides structure and support for system components. According to one embodiment, the Frame Module 110 is a rolling cart/console having locking wheels 116 as shown in FIG. 2A, to which the other subsystems may be attached and the entire system 100 can be relocated for storage. The Frame Module also provides a manually Articulating Positioning Arm 112 for holding the Localized Base Module 120 in the desired position near the operative surgical field, as shown in FIG. 2B. The Articulating Positioning Arm 112 provides a flexible positioning solution for the Localized Base Module 120. The Frame Module 110 additionally provides a Visual Display 170, which is capable of providing real time imaging for viewing during surgery. In one embodiment, the Frame Module 110 can be positioned on the non-operative side of the patient to not obstruct the surgeon during the procedure.

### Localized Base Module

As shown in FIGS. 3A-3D, the System 100 comprises a Localized Base Module (LBM) 120. The Localized Base Module 120 provides a Mounting and Communication Interface 148 to which the Kinematic Tracking Arms (KTAs) 140a, 140b attach, as shown in FIG. 3A. Proximal ends 142a, 142b of the KTAs 140a, 140b may be attached to the Mounting and Communication Interface 148, as shown in FIGS. 3C and 3D. In certain embodiments, a first kinematic tracking arm 140a may be utilized for patient tracking and a second kinematic tracking arm 140b may be used for tool tracking. In the embodiment shown in FIGS. 3A to 3D, the Localized Base Module 120 is attached to the Articulating Positioning Arm 112 connected to the Frame Module 110 and can be flexibly positioned near the surgical site. In one embodiment, the Localized Base Module 120 can be positioned anterior to the patient, avoiding the surgical field and other OR equipment. This Localized Base Module 120 establishes the system reference coordinate frame to which all other tracked entities are mapped. Primarily, it provides a Mounting and Communication Interface 148 by which the Kinematic Tracking Arms 140a, 140b attach.

In another embodiment, the Localized Base Module 120 can be provided uncoupled to the Articulating Positioning Arm 112. For example, in such an embodiment, the Localized Base Module 120 can be mounted to the OR table or to another machine or equipment.

### Kinematic Tracking Arms

As shown in FIGS. 3A and 3D, the System 100 comprises Kinematic Tracking Arms (KTAs) 140a, 140b. These KTAs provide sensor-based continuous tracking of patient and tooling position. Each of the Kinematic Tracking Arms can be attached to the Mounting and Communication Interface 148 on the Localized Base Module 120. KTAs 140a and 140b may articulate at distal ends 146a and 146b. Further, distal ends 146a and 146b may be configured to accept an end effector.

In one embodiment, proximal ends of the KTAs 142a, 142b are configured to fit into sockets of the Localized Base Module 120 and have the freedom to move about said socket. The KTA's 140a, 140b can be attached to the mounting and communication interface 148 in a ball-and-socket joint type connection. It is contemplated, however, that other electromechanical connections can also be employed.

In another embodiment, the KTAs can be removably attachable to the Localized Base Module 120, as shown in FIGS. 3B, 3C and 3D. The Mounting and Communication Interface 148 of the Localized Base Module 120 can provide a magnetic base 122 onto which the KTAs magnetically attach, for example. This allows the easy adjustment of the System 100 relative to the patient and the surgical table, providing greater flexibility to the surgeon. Of course, other non-magnetic connections are also contemplated, such as mechanical joint types.

The KTA's are provided with an electromechanical linkage that allows for 6 degrees of freedom movement of an attached end effector, which tracks the position of the patient and tooling. An end effector may be attached at a distal end 146a, 146b of the KTAs 140a, 140b. Two KTAs are provided: one for patient tracking 140a, and the other for tooling tracking 140b. The KTAs allow freedom of movement of the patient and tooling with minimal loading or restriction to movement. In some embodiments, the electromagnetic linkage of the KTA's themselves can be configure for more than 6 degrees of freedom of movement. The KTA's can employ angle position sensors for sensing technology and other arm linkage tracking technology similar to those used in standard positional metrology today. These angle position sensors allow for accurate and precise measurement of the joint angles of each link arm in the KTA; this, combined with the known geometries of the KTA arm segments and the attached components, allows for continuous determination of the KTA end effector's position in space using forward-kinematic calculations.

### Radiographic Fiducial Module & Patient Interface Module

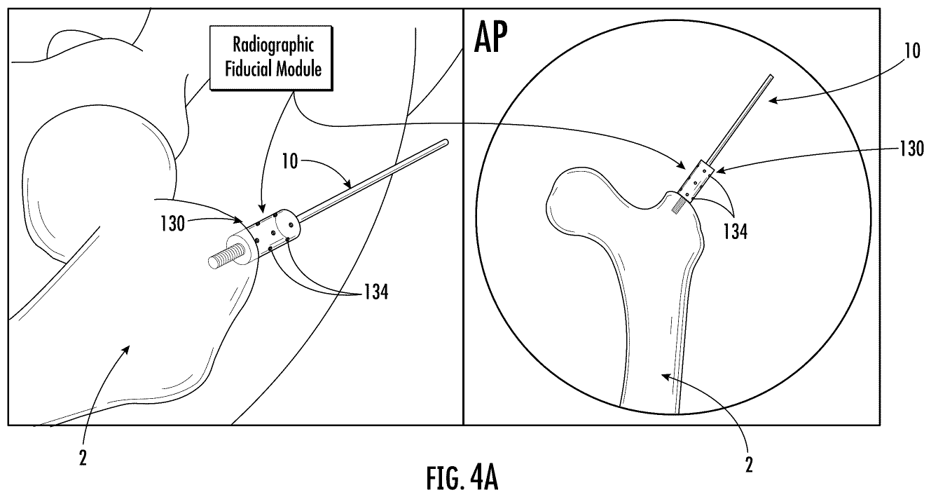

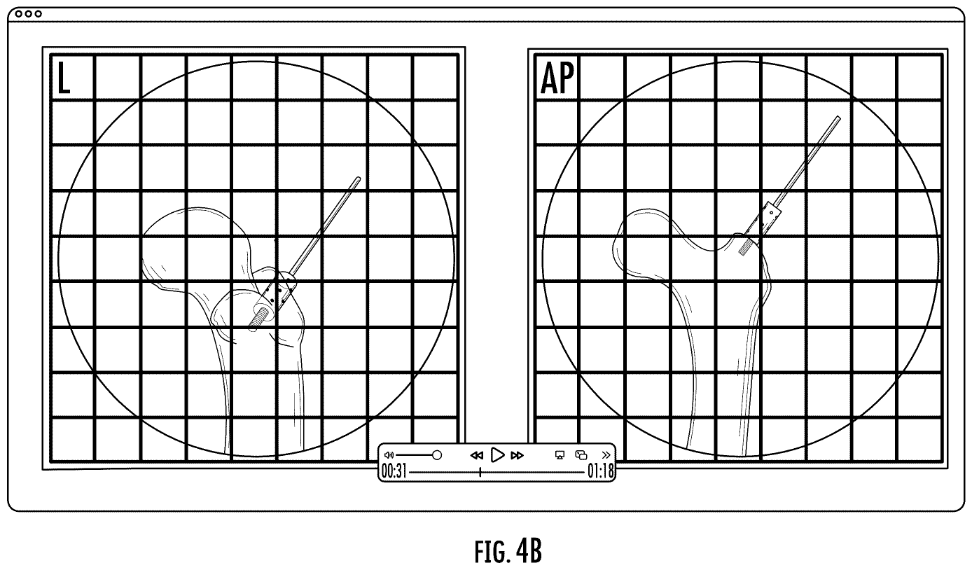

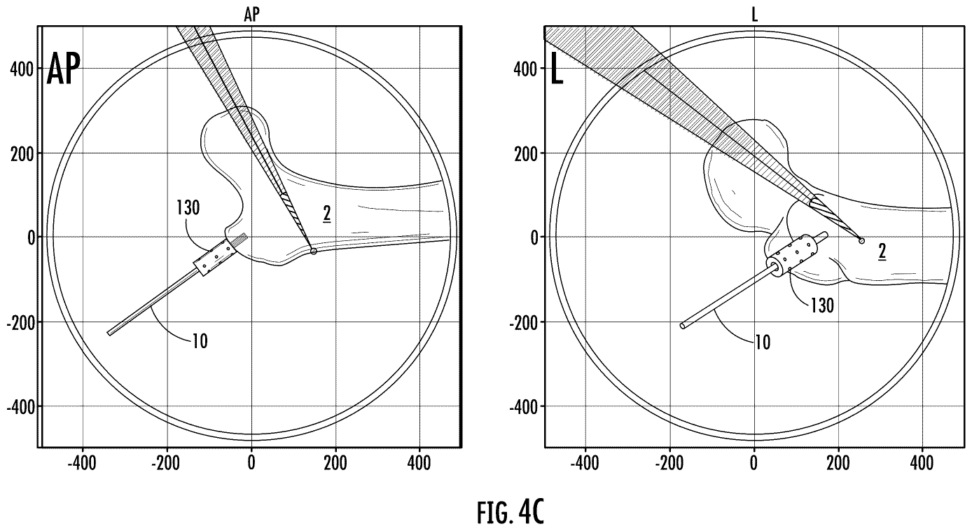

As shown in FIGS. 4A, 4B, 4C and 4D, the System 100 comprises a Patient Interface and Radiographic Fiducial Module (“RFM”) 130. The module 130 provides patient anatomy registration through fluoroscopic imaging. The module also provides rigid mechanical attachment of the patient to one of the KTAs 140a, 140b. The Radiographic Fiducial Module 130 is manufactured with an array of radiopaque markers 134 arranged in a specific and known geometry and is rigidly attached to the patient anatomy or bone 2. When captured under fluoroscopy, the radiopaque markers 134 are detectable by the image analysis algorithms implemented by the Application Software. Additionally, in the illustrated embodiment, the RFM 130 also serves as the Patient Interface Module, providing a rigid connection between the patient anatomy 2 and one of the Kinematic Tracking Arms 140a, 140b. Thus, a single module provides both functions of the RFM and the Patient Interface Module.

In an alternative embodiment, the RFM and the Patient Interface Module 130 can be provided as separate components, with a separate Radiographic Fiducial Module and a separate Patient Interface Module providing the above-described functions.

### Tooling Guide Module

As shown in FIGS. 5A and 5B, the System comprises a Tooling Guide Module 160. The Tooling Guide Module 160 guides and registers the surgical tooling (i.e., guidewires or k-wires) for position tracking. In the present embodiment, the surgical tooling is depicted as K-wire 10. The Tooling Guide Module 160 connects to a dedicated KTA 140b at its distal end 146b and establishes a known geometric relationship between the KTA end effector and the surgical tooling. For example, the precise axis of a guide wire inside the Tooling Guide Module 160 is known in 3D space due to the mechanical connection between the components. Further, a depth-tracking sensor is employed to measure the translation depth of the surgical tooling within the Tooling Guide Module 160. Thus, the 3D position of the tooling endpoint (e.g., tip of a guide wire) is also known. In one embodiment, the Tooling Guide Module 160 may contain a guide pin depth tracking encoder, microprocessor, zero depth indicator, and supplemental electrical components to aid in the tooling guide's function.

The Tooling Guide Module 160 may include a handle portion 162 to facilitate movement of the tooling when inside the Tooling Guide Module 160. The surgeon can grip the Tooling Guide Module 160 using the handle portion 162 and manipulate the position and trajectory of the tooling as desired while viewing the real-time insertion profile projection on the visual display, such as Visual Display 170 shown in FIGS. 2A and 2B.

### Distortion Correction Module

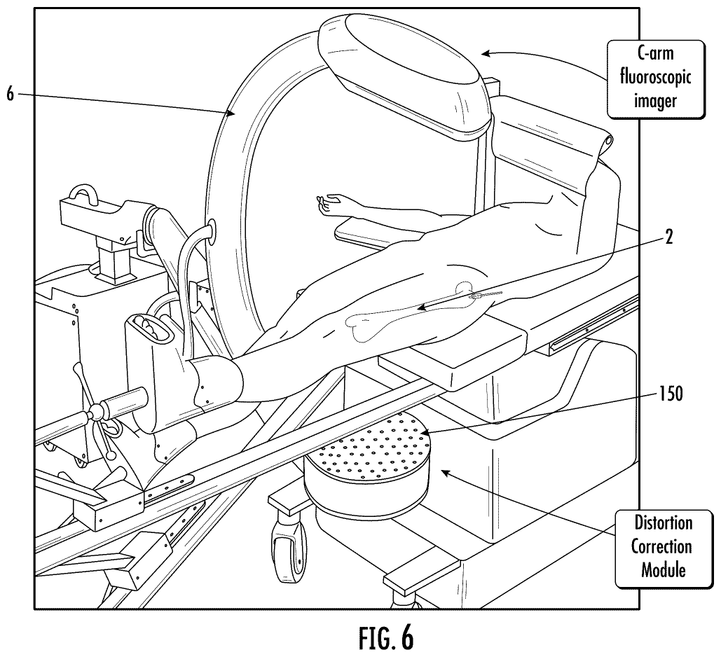

As shown in FIG. 6, the system can also include a Distortion Correction Module 150 that can be connected to a C-arm fluoroscopic imager 6. The Distortion Correction Module 150 facilitates fluoroscopic image distortion correction when capturing fluoroscopic images of patient's anatomy or bone 2. The Distortion Correction Module 150 contains an array of radiopaque points of known geometric relationship that will be present in every captured image. The System 100 uses well-established image analysis software algorithms to reverse the effects of distortion (inherent to fluoroscopic imaging processes) in the fluoroscopic images before further analysis.

### Additional System Components

Other components that can be utilized with the present System in addition to those mentioned above include:

Display Module—the Display Module 170 can be a graphic display monitor and is the primary means of communication from the System to the surgeon and OR staff. The system provides visual guidance cues and recommendations via live visualizations presented on the Display Module 130.

Computer Module—the Computer Module is a medical-grade PC for performing the computation, UI generation, image analysis, and other functions of the System.

Application Software—the Application Software represents the software components (executed on the Computer Module) for achieving the System functions. The Application Software primarily manages the logic for procedure workflows, safety and risk management, user interface and visualizations, etc.

Network Module—the Network Module represents the hardware and software components (included inside the Computer Module) for achieving connectivity to hospital data networks.

### Overview of Operation

In an exemplary process for using the System, the System 100 first registers the patient's anatomy into the System. This is accomplished through analysis of intraoperative fluoroscopic images which may optionally be combined with pre-operative CT images, when available. Once the anatomy is registered to the System 100, the System begins continuously tracking the patient anatomy's position in space; it also continually tracks the position of the Tooling Guide Module with respect to the patient anatomy. Using the known 3D positions of the anatomy and the Tooling Guide Module, a real-time presentation of position, trajectory, and tooling insertion depth (the “insertion profile”) is presented visually to the surgeon in the context of the patient's anatomy. By referencing this real-time visual display, the surgeon is enabled to modify and refine the displayed predicted insertion profile within the anatomy. Once content with the predicted insertion profile, the surgeon may begin placing the tooling into the anatomy (e.g. drilling the guide wire into the bone). As the tooling is inserted, the SGS1 also provides a live visualization of the insertion depth so that the surgeon may finalize the Tooling position with respect to the anatomical morphology as desired.

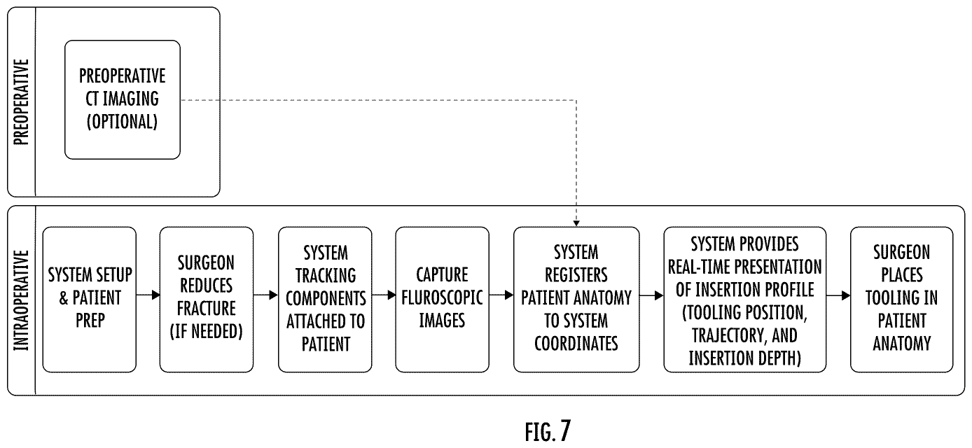

FIG. 7 represents an exemplary embodiment of a high-level surgical workflow process for using the present System 100.

In another embodiment, the operating steps of the present System 100 may also be described in the following Table 1, which represents an overview of the Operating Principles of the System.

TABLE 1
Operating Principles of the System

| Operation | Purpose | Method |
|---|---|---|
| Startup Calibration | To create a starting point | Either 1) calibrate system during |
| | with specific conditions | manufacturing or 2) require user |
| | that can be used as a | workflow of placing system |
| | reference for tracking | components into a known & repeatable |
| | changes in position. | initial position to establish a baseline. |
| Tooling position and | To measure the position | Connect a KTA (its position is known |
| orientation | and orientation of the | with respect to the Localized Base |
| measurement | Tooling & Tooling Guide | Module) to the Tooling guide, which |
| | relative to the patient's | tracks the Tooling guide with respect to |
| | anatomy | the System coordinate frame. |
| Tooling (e.g. guide | To measure the extended | An encoder attached to a friction wheel |
| wire) Depth | distance the Tooling has | contacts the Tooling, causing rotation |
| Measurement | been displaced. | of the encoder that is proportional to |
| | | the extended distance of the Tooling |
| | | (e.g. guide wire) relative to the Tooling |
| | | Guide (e.g. wire driver). A “zeroing” |
| | | functionality is provided to establish the |
| | | tip position as a baseline. |
| Kinematic Tracking | To create a starting point | Mechanically constrain to a known |
| Arm Calibration | with specific conditions | arrangement from which future |
| | that can be used as a | intraoperative movements can be |
| | reference for tracking | measured. Alternatively, calibrate to a |
| | changes in position of the | known position during device |
| | Kinematic Tracking Arm. | manufacturing which is persistent |
| | | across system power cycles. |
| Anatomy | To determine the | Utilize fluoroscopic images and the |
| Registration | orientation and position of | known geometries of the Radiographic |
| to System | the Radiographic Fiducial | Fiducial Module to register the anatomy |
| | Module with respect to | to the coordinate system |
| | the anatomy of interest. | |
| Anatomy Movement | To determine the | Connect the Kinematic Tracking Arm |
| Measurement | anatomical position with | (its position is known with respect to |
| | respect to the System | the Localized Base Module) to the |
| | coordinate frame | Patient Interface Module (same |
| | | component as Radiographic Fiducial |
| | | Module); and monitor all changes in |
| | | position throughout the procedure, |
| | | updating the visualizations to remain |
| | | accurate in response. |
| Navigation Display | Provide visual navigation | Project tooling (e.g. guide wire) |
| | to surgeon during tooling | position and trajectory onto |
| | use | fluoroscopic images, showing the |
| | | projected insertion path into the |
| | | anatomy. Note: System does not |
| | | attempt to create a 3D reconstruction |
| | | of the patient's anatomy using 2D |
| | | fluoroscopic images. Rather, a 2D-to- |
| | | 3D point reconstruction technique is |
| | | employed to register the patient's |
| | | anatomy to 3D coordinates into the |
| | | System coordinate frame. |
| Fluoroscopic Image | To overcome image | The Distortion Correction Module |
| Distortion Correction | distortion inherent to | (containing a known arrangement of |
| | fluoroscopic imaging. | radiographically visible points) is |
| | | placed on the C-arm image intensifier. |
| | | Established image analysis algorithms |
| | | are used to reverse the effects of |
| | | distortion before further image analysis. |
| Point Projection | Accurately depict tooling | The radiographic fiducial module is |
| | insertion profile as an | rigidly attached to the patient's |
| | overlay on fluoroscopic | anatomy and contains radiopaque |
| | images. | spheres with known geometric |
| | | relationships. System identifies these |
| | | points in analyzed images and |
| | | determines the fiducial module's |
| | | orientation and scaling. The fiducial |
| | | module's pose with respect to the |
| | | tracked tooling is known at all times |
| | | through the tracking system. |
| | | While the 3D contours of the anatomy |
| | | are cannot be determined using only |
| | | fluoroscopic images by the System, it |
| | | can project any other known point in |
| | | 3D space accurately onto the existing |
| | | image, including the projected tooling |
| | | path within the anatomy. |

Additionally, the System 100 may be configured to perform data collection for use in various analyses, such as the following:
1. Data Collection and ML Model building with various data inputs
2. Computer Vision and Medical Image processing
   a. Convolutional Neural Network (or other methods) for deriving 3D bone structure from minimal set of 2D x-rays
   b. Bone quality assessment and identification of highest quality regions
   c. Bone fragment segmentation and position identification
3. Performance and procedural efficiency assessment from users
   a. Closed reduction evaluation
   b. Procedural step performance evaluation
   c. Motion economy and procedural efficiency assessments
4. Placement guidance derived from user validated models
   a. Optimal placement locations within target structure
   b. Implant selection and sizing optimization
   c. Implant configuration
5. Procedural stage classification

It is further contemplated that the present System 100 may be configured to utilize artificial intelligence (AI) to exploit the data collected and to perform these analyses.

Further, while the present System is described for use in inserting a guidewire or k-wire, it is contemplated that the present system can be configured for other orthopedic surgical techniques and procedures, such as for example, for use with a drill bit or reamer to drill a hole into bone, or for bone cutting or bone debridement, as well as for use in dental and non-orthopedic surgical applications.

Other embodiments will be apparent to those skilled in the art from consideration of the specification and practice of the embodiment disclosed herein. It is intended that the specification and examples be considered as exemplary only, with a true scope and spirit of the embodiment being indicated by the following claims.

For example, in a first aspect, a 1^{st} embodiment of a system for surgical guidance and navigation for tracking a tooling position relative to a patient is provided. The system comprises: a frame module housing a computerized subsystem including a computer module therein, and having electronically connected thereto a visual display; an articulating positioning arm connected to the frame module; a localized base module in communication with the frame module; and first and second kinematic tracking arms attached to the localized base module, the first kinematic tracking arm being configured to connect to a patient's bone for patient tracking, and the second kinematic tracking arm being configured to receive a surgical tooling for tooling tracking.

A 2^{nd} embodiment is the 1^{st} embodiment, wherein the localized base module is attached to the articulating positioning arm and is configured to communicate with the computerized subsystem of the frame module.

A 3^{rd} embodiment is any combination of the above embodiments, wherein localized base module is configured to serve as a reference coordinate frame to which all other tracked entities are mapped.

A 4^{th} embodiment is any combination of the above embodiments, wherein the localized base module includes a mounting and communication interface for connection to the first and second kinematic tracking arms.

A 5^{th} embodiment is any combination of the above embodiments, wherein the mounting and communication interface is configured to receive the first and second kinematic tracking arms.

A 6^{th} embodiment is any combination of the above embodiments, wherein the first and second kinematic tracking arms are removably attachable to the mounting and communication interface of the localized base module.

A 7^{th} embodiment is any combination of the above embodiments, wherein the first and second kinematic tracking arms are configured for sensor-based continuous tracking.

An 8^{th} embodiment is any combination of the above embodiments, wherein the first and second kinematic tracking arms include at least one encoder configured to provide data of a measurement of at least one joint in in the first and second kinematic tracking arms.

A 9^{th} embodiment is any combination of the above embodiments, wherein the first and second kinematic tracking arms each include an electromechanical linkage.

A 10^{th} embodiment is any combination of the above embodiments, wherein the electromechanical linkage is configured to provide 6 degrees of freedom movement of an attached end effector.

An 11^{th} embodiment is any combination of the above embodiments, further including a radiographic fiducial module for connection to a distal end of one of the first or second kinematic tracking arms.

A 12^{th} embodiment is any combination of the above embodiments, wherein the radiographic fiducial module is configured to attach to the patient's bone.

A 13^{th} embodiment is any combination of the above embodiments, wherein the radiographic fiducial module comprises an array of radiopaque markers arranged in a geometric configuration.

A 14^{th} embodiment is any combination of the above embodiments, further including a patient interface module for connection to a distal end of one of the first or second kinematic tracking arms.

A 15^{th} embodiment is any combination of the above embodiments, wherein the patient interface module is configured to attach to the patient's bone.

A 16^{th} embodiment is any combination of the above embodiments, further including a tooling guide module for connection to a distal end of the second kinematic tracking arm.

A 17^{th} embodiment is any combination of the above embodiments, wherein the tooling guide module is configured to receive a surgical tooling.

An 18^{th} embodiment is any combination of the above embodiments, wherein the surgical tooling comprises a guidewire, drill bit, reamer, or saw blade.

A 19^{th} embodiment is any combination of the above embodiments, wherein the tooling guide module includes a depth-tracking sensor for determining a translation depth of the guidewire.

A 20^{th} embodiment is any combination of the above embodiments, wherein the tooling guide module further includes a guide pin depth tracking encoder, a microprocessor, or a zero depth indicator individually or any combination thereof.

A 21^{st} embodiment is any combination of the above embodiments, wherein the tooling guide module includes a handle portion configured to allow manipulation of the position and trajectory of a tooling received within the tooling guide module.

A 22^{nd} embodiment is any combination of the above embodiments, wherein the first and second kinematic tracking arms are independently movable with respect to one another.

A 23^{rd} embodiment is any combination of the above embodiments, further including a distortion correction module.

A 24^{th} embodiment is any combination of the above embodiments, wherein the distortion correction module includes an array of radiopaque points arranged in a geometric configuration.

A 25^{th} embodiment is any combination of the above embodiments, further being configured to provide real-time insertion profile projection of the tooling into a patient's bone on the visual display.

A 26^{th} embodiment is any combination of the above embodiments, wherein the frame module includes a plurality of locking wheels positioned along a base of the frame module.

In another aspect, in a 1^{st} embodiment, a method of performing orthopedic surgery using the surgical guidance and navigation system of the above embodiments is provided. The method comprises: attaching the localized base module to the articulating positioning arm; attaching the first and second kinematic tracking arms to a mounting and communication interface of the localized base module; attaching a tooling guide module to a distal end of the second kinematic tracking arm; inserting a surgical tool into the tooling guide module; attaching a radiographic fiducial model and a patient interface module to a distal end of the first kinematic tracking arm; positioning the frame module relevant to a patient prepared for surgery; positioning the articulating positioning arm such that the localized base module is located anterior to the patient; activating the computerized subsystem of the frame module; initiating a startup calibration routine to establish a baseline and repeatable initial position; securing the patient interface module to a bone of the patient; capturing fluouroscopic images; tracking the kinematic tracking arms and at least one end effector using known geometries and precise measurements of at least one joint angle of each arm to conduct forward kinematics calculations; detecting a radiopaque marker of the radiographic fiducial model to provide an image of a surgical worksite; measuring a translation depth of the surgical tool with a depth tracking sensor installed in the tooling guide module; and providing a real-time presentation of an insertion profile of the surgical tool.

A 2^{nd} embodiment is the 1^{st} embodiment, further including the step of conducting pre-operative CT imaging prior to attaching the localized base module to the articulating positioning arm.

A 3^{rd} embodiment is any combination of the above embodiments, further including the step of inserting a guidewire into a patient's bone using the system of the above embodiments.

A 4^{th} embodiment is any combination of the above embodiments, further including the step of determining a desired depth and trajectory of the guidewire into the patient's bone.

A 5^{th} embodiment is any combination of the above embodiments, wherein the step of measuring a translation depth of the surgical tool further comprises viewing an image depicting a position of the surgical tool in 3D space displayed on the visual display of the frame module.

A 6^{th} embodiment is any combination of the above embodiments, further including the step of utilizing a distortion correction module to correct fluoroscopic image distortion.

In yet another aspect, in a 1^{st} embodiment, a method of performing orthopedic surgery using the surgical guidance and navigation system of the above embodiments is provided. The method comprises: taking pre-operative CT images; registering patient anatomy to the system; continuously tracking a spatial position of the patient anatomy; continuously tracking a spatial position of a tooling guide module in relation to the patient anatomy; presenting a real-time visual display of an insertion profile comprising a position, trajectory, and tooling insertion depth of a surgical tool; modifying and refining the displayed insertion profile within the patient anatomy; placing the surgical tool within the patient anatomy; and manipulating the surgical tool in accordance with the real-time display of the insertion profile to achieve a desired position of the surgical tool.

What is claimed is:

1. A surgical guidance and navigation system for tracking a tooling position relative to a patient, the system comprising:
a frame module housing a computerized subsystem including a computer module therein, and having electronically connected thereto a visual display;
an articulating positioning arm connected to the frame module;
a localized base module in communication with the frame module; and
first and second kinematic tracking arms attached to the localized base module, the first kinematic tracking arm being configured to connect to a patient's bone for patient tracking, and the second kinematic tracking arm being configured to receive a surgical tooling for tooling tracking.

2. The system of claim 1, wherein the localized base module is attached to the articulating positioning arm and is configured to communicate with the computerized subsystem of the frame module.

3. The system of claim 1, wherein localized base module is configured to serve as a reference coordinate frame to which all other tracked entities are mapped.

4. The system of claim 1, wherein the localized base module includes a mounting and communication interface for connection to the first and second kinematic tracking arms.

5. The system of claim 4, wherein the mounting and communication interface is configured to receive the first and second kinematic tracking arms.

6. The system of claim 5, wherein the first and second kinematic tracking arms are removably attachable to the mounting and communication interface of the localized base module.

7. The system of claim 1, wherein the first and second kinematic tracking arms are configured for sensor-based continuous tracking.

8. The system of claim 7, wherein the first and second kinematic tracking arms include at least one encoder configured to provide data of a measurement of at least one joint in in the first and second kinematic tracking arms.

9. The system of claim 1, wherein the first and second kinematic tracking arms each include an electromechanical linkage.

10. The system of claim 9, wherein the electromechanical linkage is configured to provide 6 degrees of freedom movement of an attached end effector.

11. The system of claim 1, further including a radiographic fiducial module for connection to a distal end of one of the first or second kinematic tracking arms.

12. The system of claim 11, wherein the radiographic fiducial module is configured to attach to the patient's bone.

13. The system of claim 11, wherein the radiographic fiducial module comprises an array of radiopaque markers arranged in a geometric configuration.

14. The system of claim 1, further including a patient interface module for connection to a distal end of one of the first or second kinematic tracking arms.

15. The system of claim 14, wherein the patient interface module is configured to attach to the patient's bone.

16. The system of claim 1, further including a tooling guide module for connection to a distal end of the second kinematic tracking arm.

17. The system of claim 12, wherein the tooling guide module is configured to receive a surgical tooling.

18. The system of claim 17, wherein the surgical tooling comprises a guidewire, drill bit, reamer, or saw blade.

19. The system of claim 16, wherein the tooling guide module includes a depth-tracking sensor for determining a translation depth of the guidewire.

20. The system of claim 16, wherein the tooling guide module further includes a guide pin depth tracking encoder, a microprocessor, or a zero depth indicator individually or any combination thereof.

21. The system of claim 16, wherein the tooling guide module includes a handle portion configured to allow manipulation of the position and trajectory of a tooling received within the tooling guide module.

22. The system of claim 1, wherein the first and second kinematic tracking arms are independently movable with respect to one another.

23. The system of claim 1, further including a distortion correction module.

24. The system of claim 23, wherein the distortion correction module includes an array of radiopaque points arranged in a geometric configuration.

25. The system of claim 1, further being configured to provide real-time insertion profile projection of the tooling into a patient's bone on the visual display.

26. The system of claim 1, wherein the frame module includes a plurality of locking wheels positioned along a base of the frame module.
